(19) United States
(12) Patent Application Publication
(10) Pub. No.: US 2007/0032462 A1
(43) Pub. Date: Feb. 8, 2007

(54) Novel methods for the preparation of dhea derivatives

(76) Inventors: Marc Criton, Montpellier (FR); Damien Gloux, Montpellier (FR); Jean-Louis Montero, Valflaunes (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/552,336

(22) PCT Filed: Apr. 6, 2004
(86) PCT No.: PCT/FR04/00847

§ 371 Date: Jul. 13, 2006

(30) Foreign Application Priority Data
Apr. 7, 2003 (FR) 030482

(51) Int. Cl. C07J 1/00; A61K 31/56

(52) U.S. Cl. 514/169; 552/509

(57) ABSTRACT

The invention relates to a method for the production of DHEA derivatives, such as 7-oxo-DHEA and 7-hydroxy-DHEA, from DHEA itself.

The invention relates to novel methods for preparing DHEA derivatives from DHEA itself.

The invention relates in particular to novel methods for preparing 7α-hydroxydehydroepiandrosterone (7α-OH-DHEA), 7β-hydroxydehydroepiandrosterone (7β-OH-DHEA), and 7-oxo-dehydroepiandrosterone (7-oxo-DHEA).

DHEA is a natural steroid produced essentially by the adrenocortical glands. DHEA administered topically or orally is known for its ability to promote epidermal keratinization (JP-07 196 467).

Moreover, its role in combating the weathered appearance of the skin (FR 00/00349), its ability to modulate the pigmentation of the skin and of the hair (FR 99/12773) and its action against epidermal atrophy (FR 00/06154) have been demonstrated. Although these properties make it a candidate of choice as a cosmetic or dermatological active agent, the therapeutic use of DHEA has revealed adverse side effects, in particular in women, as a potential precursor of androgenic hormones.

Among DHEA metabolites, particular attention has been given in recent years to 7(α and β)-OH-DHEA and to 7-oxo-DHEA. This is because it has been demonstrated that these metabolites are devoid of the hormonal effect of DHEA and exhibit advantageous pharmacological and cosmetic actions. The 7-hydroxylated derivatives increase fibroblast proliferation and keratinocyte viability and have a free-radical scavenger activity (WO 98/40074).

Several documents describe the production of 7α-OH-DHEA by the chemical process and its use in the treatment of Alzheimer's disease (WO 94/03176), as an agent for stimulating immunity (WO 93/20696, WO 94/031765, WO 94/08588, WO 96/35428), as an anti-glucocorticoid agent (WO 94/08588), for the treatment of obesity (WO 92/03925), and for the treatment of diabetes and of certain cancers (U.S. Pat No. 4,898,694).

7-oxo-DHEA itself also has pharmacological and cosmetic properties, but without the hormonal effects of DHEA. It has been described as being effective in the modulation of the immune system (U.S. Pat. No. 5,292,730; U.S. Pat. No. 5,585,371; U.S. Pat. No. 5,641,766), the treatment of Alzheimer's disease (U.S. Pat. No. 5,707,983) and the treatment of HIV syndrome (U.S. Pat. No. 5,885,977), and for promoting weight loss (U.S. Pat. No. 5,296,481 and U.S. Pat. No. 5,807,848).

In addition, document WO 99/25333 mentions the topical use of 7-oxo-DHEA for the prophylactic and curative treatment of lupus erythematosus.

Moreover, the pharmacological properties of these compounds in dermatology and/or in cosmetics have led many teams to use them in combination with bioactive molecules that are used in cosmetics, such as α-hydroxy acids, β-hydroxy acids, α-keto acids, β-keto acids and retinoids (FR 2 799 649-A1 and FR 2 818 133-A1).

These various data clearly show that it would be advantageous to provide novel methods for obtaining 7α-OH-DHEA, 7β-OH-DHEA and 7-oxo-DHEA.

Among the documents that relate a method of synthesizing 7-OH-DHEA, mention may be made of: FR 2 771 105-A1, FR 2 793 491-A1, WO-A1 94/03176, WO-A1-92/03925 and FR 2 820 745-A1.

Document FR 2 771 105-A1 describes a method for preparing 7α-OH-DHEA by bioconversion using Fusarium moniliforme. However, the toxins secreted by the latter can prove to be very dangerous for humans.

The subject of document FR 2 793 491-A1 is the production of 7α-OH-DHEA in two steps. The allylic oxidation of 3-O-acetyl-DHEA using copper catalysts and a perester (tert-butyl perbenzoate) gives the diester (3β-acetate, 7α-benzoate dehydroxyandrost-5-ene-17-one) in the form of the stereospecifically pure α-isomer. The latter, after treatment with sodium methanolate, gives 7α-OH-DHEA.

Documents WO-A1-94/03176 and WO-A1-92/03925 describe a 4-step method using the 3β-acetate of DHEA. This method consists of an allylic bromination, which gives the unstable mixture of the 7α- and 7β-bromo isomers. Said bromination is followed by selective isomerization in favor of the 7α-bromo DHEA isomer, which is subsequently hydrolyzed using acetic acid and silver acetate so as to give, after deprotection of the 3-position, 7α-OH-DHEA.

Document FR 2 820 745-A1 provides 3-step and 5-step methods of synthesis. The 3-step synthesis consists in carrying out an oxidation on the allylic position of 3-O-acetyl-DHEA, so as to obtain the 3-acetylated derivative of 7-oxo-DHEA.

Document WO 03/02124 describes DHEA derivatives of the bis(5-androstene-17-one-3β-hydroxy) acid diester type and their use for the treatment of myocardial ischemia, arrhythmia, cerebral ischemia, hypoimmunity, osteoporosis, etc. However, that document in no way envisions the use of these diesters for the synthesis of 7α-OH-DHEA, 7β-OH-DHEA and 7-oxo-DHEA.

There remains a need for a method for obtaining 7α-OH-DHEA, 7β-OH-DHEA and 7-oxo-DHEA which has better yields compared with the methods of the prior art.

It is sought to obtain these products by means of a simple synthetic process that can be readily extrapolated to an industrial scale, giving products that are easy to purify. In addition, in the case of 7α-hydroxy-DHEA and 7β-hydroxy-DHEA, the desire is to be able to obtain one or other product with a satisfactory diastereoselectivity but without requiring a step to eliminate the minority diastereoisomer.

This is the aim of the present invention, the subject of which is novel methods for synthesizing DHEA derivatives, these methods using DHEA as starting product.

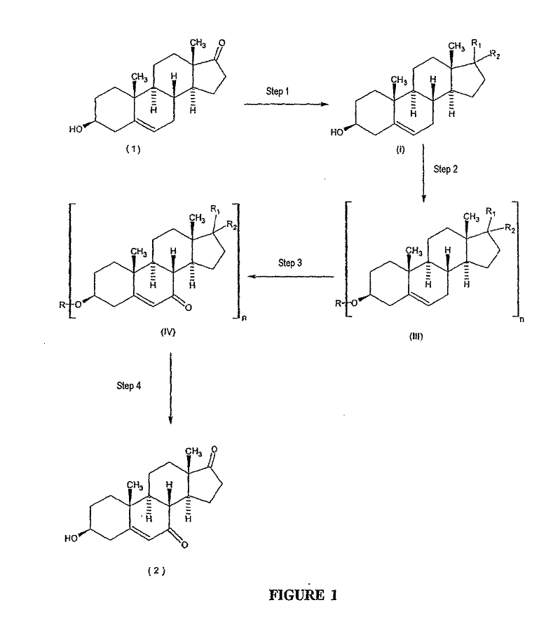

FIG. 1 illustrates a method that makes it possible to obtain 7-oxo-DHEA in four steps, from DHEA:

(1) in a first, optional step, the ketone function in the 17-position of DHEA is optionally protected with an appropriate protective group.

The compound of formula (I) is obtained: 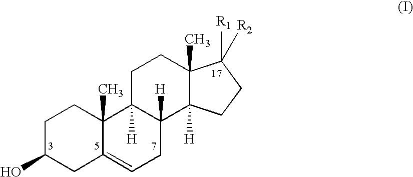
in which R₁, R₂, taken together, represent a group ═O (in the case where the ketone function is not protected), or R₁ and R₂, taken together, represent a group —O—W—O—, in which W represents a saturated or unsaturated, linear, branched or cyclic C₂-C₈ alkyl group;

(2) in a second step, the hemiester (n=1) or the diester (n=2) is prepared between the alcohol in the 3-position of the compound (I) and a diacid chosen from those corresponding to formula (II):
HOOC—X—COOH (II)
in which X represents a single bond or a group chosen from —CH₂—, saturated or unsaturated, linear, branched or cyclic C₂-C₂₀ alkyls, C₆-C₂₀ aryls and C₈-C₂₀ aralkyls. This (hemi- or di-) ester corresponds to the formula (III): 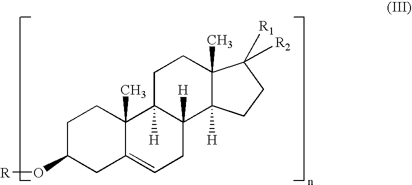
in which n, R₁ and R₂ have the same meaning as above, and either R represents —OC—X—CO— (in the case where n=2) or R represents HOOC—X—CO (in the case where n=1);

(3) in a third step, an allylic oxidation is carried out on the carbon in the 7-position, so as to obtain the ketone of formula (IV): 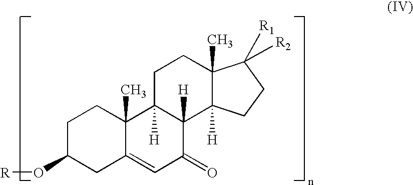
in which n, R₁, R₂ and R have the same meaning as above;

(4) in a fourth step, the protective groups are removed from the ketone in the 17-position (optionally) and from the alcohol in the 3-position, so as to obtain the 7-oxo-DHEA (2).

When it is desired to simply obtain the 7-oxo-DHEA, the protection of the ketone in the 17-position (step 1) and its subsequent deprotection (step 4), are not necessary.

The method of the invention differs from the methods of the prior art in particular in that a hemiester or a diester (III) of a diacid of formula (II) and of DHEA are used to prepare the 7-oxo-DHEA, 7α-hydroxy-DHEA and 7β-hydroxy-DHEA.

The use of these esters (III) has several advantages compared with the compounds of the prior art:
better oxidation yields;
intermediate products that are easier to purify, these products generally being obtained by recrystallization;
the extension of the chain in the 3-position hinders the β-face and makes it possible to increase the diastereoselectivity of the reduction of 7-oxo and 7β-hydroxy derivatives with NaBH₄ without necessarily adding cerium chloride (CeCl₃);
when R represents HOOC—X—CO, in the context of a supported synthesis strategy, this group can be readily attached to a solid support. This methodology has the advantage of promoting the elimination of excess reagents and therefore of facilitating the purification step.

According to the invention, the DHEA (1) is first of all optionally protected in the 17-position, preferably in the form of a cyclic acetal.

For example, R₁, R₂=—O—CH₂—CH₂—O—.

Advantageously, the DHEA (1) is treated with ethylene glycol, at the reflux of toluene, in the presence of para-toluenesulfonic acid using a Dean Stark apparatus. The protected product is obtained by recrystallization from an alcoholic solution (methanol or ethanol, for example).

According to another variant of the invention, the acetalization of the DHEA, in the 17-position, can be omitted. In this case, R₁, R₂, taken together, represent ═O. This is in particular the case when the synthesis stops at the oxidation in the 7-position of the DHEA.

In general, the second step of the method represented by FIG. 1 is carried out, when n=1, by treatment of a compound of formula (I) with a molar equivalent of a dicarboxylic acid, in acid form or in an activated form, such as acid chloride or acid anhydride.

Among the dicarboxylic acids that can be used in the present invention, mention may, for example, be made of:

succinic acid, glutaric acid, suberic acid, maleic acid and phthalic acid, which are advantageously used in the form of anhydrides; oxalic acid, which is advantageously used in the form of the chloride;

isophthalic acid, terephthalic acid, 1,2-phenyl-enediacetic acid, 1,3-phenylenediacetic acid and 1,4-phenylenediacetic acid, which are advantageously used in mono acid chloride form.

When n=2, the process is carried out in the same way, using 2 molar equivalents of DHEA derivative (I) per mole of diacid (II).

In any case, the product (III) is purified by silica gel column chromatography.

The products corresponding to formula (III) in which
n=1, 2,
R₁, R₂ represent ═O or a group —O—W—O in which W represents a saturated or unsaturated, linear, branched or cyclic C₂-C₈ alkyl group; preferably, W represents —CH₂—CH₂—,
R represents a group of formula (IIa)
   (HO)ₘOC—(X)—CO— (IIa)
   in which

m represents 0 when n=2 and m represents 1 when n=1,

X represents a single bond or a group chosen from —(CH₂)—, saturated or unsaturated, linear, branched or cyclic C₂-C₂₀ alkyls, C₆-C₂₀ aryls and C₈-C₂₀ aralkyls, it being understood that, when n=1, X is different from —(CH₂)₂— and from —(CH₂)₆— and that, when n=2, X is different from —(CH₂)—, from —(CH₂)₂—, from —(CH₂)₃— and from —(CH₂)₄—,

are novel products and constitute another subject of the invention. These products are intermediates that make it possible to obtain DHEA derivatives with good yields and a readily industrializable method.

The third step of the method illustrated by FIG. 1 is the oxidation in the 7-position (allylic oxidation) of the DHEA ring.

Allylic oxidation is a reaction that is well known in organic chemistry. Some of the methods used suffer from low yields, from tricky working conditions (reaction conditions, t⁰, treatment, etc.), from the use of expensive and/or ecologically and physiologically undesirable reactants, such as chromium.

Thus, the present invention provides a method of oxidation in an organic solvent using oxygen, photons and a photochemical sensitizing agent.

The compounds of formula (III) undergo allylic oxidation by photooxidation by means of a lamp and with sparging with oxygen (or with compressed air) in the presence of rose Bengal.

A sodium lamp is preferably used. The corresponding 5-hydroperoxide is obtained, which is readily converted to 7-hydroperoxide and then to 7-oxo by treatment with CuCl₂ in pyridine or, advantageously, with acetic anhydride in pyridine, as illustrated in scheme 1 below.

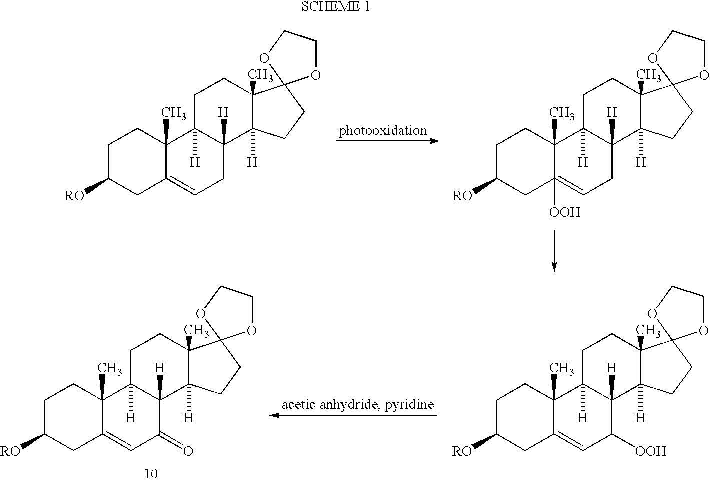

Moreover, according to a variant of the invention, a second form of execution of the oxidation reaction in the allylic position of the compounds of formula (III) can be carried out using N-hydroxyphthalimide with sparging with oxygen or with compressed air, as described in documents U.S. Pat. No. 5,030,739 and “Steroids”, 1998, 63(3), pp 158-165.

The products corresponding to formula (IV) in which
n=1, 2,
R₁, R₂ represent ═O or a group —O—W—O— in which W represents a saturated or unsaturated, linear, branched or cyclic C₂-C₈ alkyl group; preferably, W represents —CH₂—CH₂—,
R represents a group of formula (IIa)
   (HO)ₘ—OC—(X)—CO— (IIa)
   in which:

m represents 0 when n=2 and m represents 1 when n=1,

X represents a single bond or a group chosen from —(CH₂)—, saturated or unsaturated, linear, branched or cyclic C₂-C₂₀ alkyls, C₆-C₂₀ aryls and C₈-C₂₀ aralkyls, are novel products and constitute another subject of the invention.

The compound (IV) can subsequently be deprotected in a conventional manner by treatment with sodium methanolate in methanol (deprotection of the protective group R of the alcohol in the 3-position) and, optionally, treatment with a perchloric acid solution (deprotection of the ketone in the 17-position when said ketone has been protected).

The 7-oxo-DHEA (2) is thus obtained.

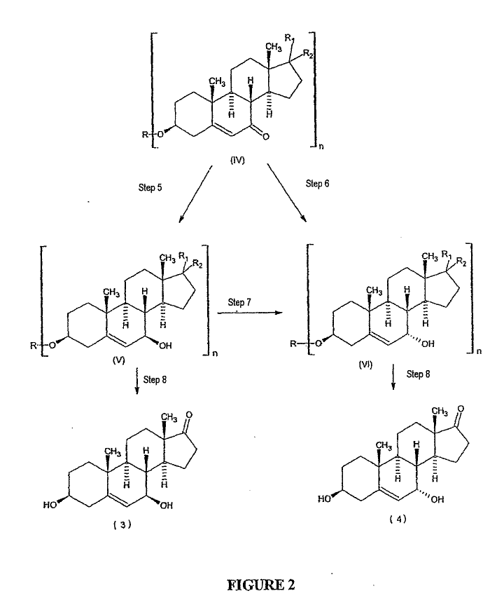

According to a variant of the invention illustrated by FIG. 2, the ketone of formula (IV) can be used to prepare the 7α-OH-DHEA (4) and the 7β-OH-DHEA (3).

The compounds of formula (IV) in which the 17-position is protected with an acetal (R₁, R₂ represent —O—W—O—) can be diastereoselectively reduced with lithium tri-sec-butylborohydride, known under the commercial name L-Selectride®, at low temperature, so as to give the corresponding 7α-OH derivatives that correspond to formula (VI) (step 6). The 17-position can subsequently be deprotected in a known manner.

The products corresponding to formula (VI): 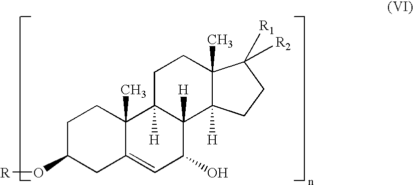
in which
n=1, 2,
R₁, R₂ represent ═O or a group —O—W—O— in which W represents a saturated or unsaturated, linear, branched or cyclic C₂-C₈ alkyl group; preferably, W represents —CH₂—CH₂—,
R represents a group of formula (IIa)
   (HO)ₘ—OC—(X)—CO— (IIa)
   in which:

m represents 0 when n=2 and m represents 1 when n=1,

X represents a single bond or a group chosen from —(CH₂)—, saturated or unsaturated, linear, branched or cyclic C₂-C₂₀ alkyls, C₆-C₂₀ aryls and C₈-C₂₀ aralkyls, are novel products and constitute another subject of the invention.

The compounds of formula (VI) are deprotected in 3-position by trans-esterification by treatment with sodium methanolate in methanol.

The deprotection of the acetal in the 17-position is carried out by treatment with a perchloric acid solution and makes it possible to obtain the 7α-OH-DHEA (4).

According to another variant, the compounds of formula (IV), and in which the 17-function is protected (R₁, R₂ represent —O—W—O—) can be diastereoselectively reduced by treatment with NaBH₄ in the presence of cerium chloride, so as to give the 7β-hydroxylated derivatives of formula (V) (step 5). The 17-position can subsequently be deprotected in a known manner.

The products corresponding to formula (V): 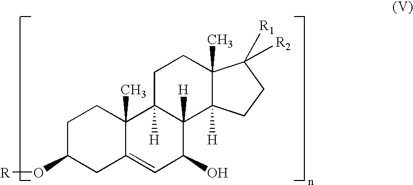
in which
n=1, 2,
R₁, R₂ represent ═O or a group —O—W—O— in which W represents a saturated or unsaturated, linear, branched or cyclic C₂-C₈ alkyl group; preferably, W represents —CH₂—CH₂—,
R represents a group of formula (IIa)
   (HO)ₘ—OC—(X)—CO— (IIa)
   in which:

m represents 0 when n=2 and m represents 1 when n=1,

X represents a single bond or a group chosen from —(CH₂)—, saturated or unsaturated, linear, branched or cyclic C₂-C₂₀ alkyls, C₆-C₂₀ aryls and C₈-C₂₀ aralkyls, are novel products and constitute another subject of the invention.

Deprotection of the acetal in the 17-position and of the ester in the 3-position gives the 7β-OH-DHEA (3).

According to yet another variant of the invention, the compounds of formula (V) can undergo an inversion of configuration so as to give the 7α-hydroxy or ester derivatives of formula (VI), and vice versa. To do this, the present invention provides two pathways:

The first pathway, according to an application of the Mitsunobu reaction, implements an inversion of configuration of the 7β-hydroxyl to 7α-hydroxyl, or vice versa, using the diethyl azodicarboxylate/tri-phenylphosphine system and carboxylic acid (and, more particularly, para-nitrobenzoic acid) or the system of N,N,N′,N′-tetramethylazodicarboxamide and tributyl phosphine in the presence of para-methoxybenzoic acid.

These systems are chosen so as to promote the inversion reaction. Finally, subsequent deprotection in a methanolate medium and then perchloric acid makes it possible to deprotect the 3- and 17-positions.

More generally, the para-methoxybenzoic acid can be replaced with a carboxylic acid chosen from those corresponding to the formula R₅CO₂H where R₅ can be chosen from the compounds corresponding to the formula R₄Ph, R₄ being chosen from H—, NO₂—, CH₃O—, CN—, Cl—, Br— and F—, and R₅ can also be chosen from the group consisting of: CH₃—, ClCH₂—, Cl₂CH—, Cl₃C— and CH₃CH₂—.

Another pathway for preparing 7α-OH-DHEA by inversion of 7β derivatives can be used in the method of the invention. It consists in introducing a good leaving group in the 7-position of the 7β-OH, using methanesulfonyl chloride (MsCl), para-toluenesulfonyl chloride (TsCl) or a trifluoromethanesulfonyl chloride (TfCl), and then carrying out a Walden inversion by nucleophilic substitution of SN₂ type in a basic medium (OH—) (step 7).

Alternatively, the leaving group can be displaced with the alkali metal salt of a carboxylic acid (cesium, sodium or potassium) so as to give the corresponding diester. The carboxylic acid will be chosen from compounds corresponding to the formula R₅CO₂H, where R₅ can be chosen from the compounds corresponding to the formula R₄Ph, in which R₄ may be chosen from H—, NO₂—, CH₃O—, CN—, Cl—, Br— and F—, and R₅ can also be chosen from the group consisting of: CH₃—, ClCH₂—, Cl₂CH—, Cl₃C— and CH₃CH₂—.

Under these conditions, the reaction may be carried out conventionally or may be accelerated by ultrasonic activation.

The intermediate compounds in the Mitsunobu reactions can be represented by formulae Va and VIa below: 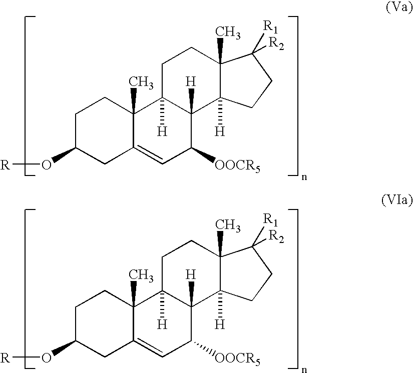
in which the groups R, R₁, R₂ and R₅ and the integer n have the same meaning as above. They are also novel compounds that constitute another subject of the invention.

The compounds of formula (VI) are deprotected by trans-esterification by treatment with sodium methanolate in methanol (step 8).

The deprotection of the carbonyl in the 17-position, protected with an acetal, is carried out by treatment with a perchloric solution and thus makes it possible to obtain the 7α-OH-DHEA.

All the compounds of formula (III), (IV), (V), (Va), (VI) and (VIa), and also the 7α-OH-DHEA, the 7β-OH-DHEA and the 7-oxo-DHEA, can be included in a single formula (A): 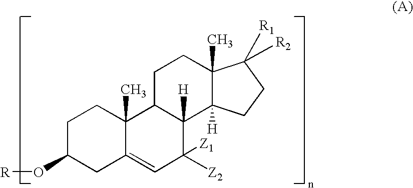
in which
n=1, 2,
R₁, R₂ represent ═O or a group —O—W—O— in which W represents a saturated or unsaturated, linear, branched or cyclic C₂-C₈ alkyl group; preferably, W represents —CH₂—CH₂—,
Z₁, Z₂ represent ═O, (H,OH) or (H,H), (H, R₅CO₂—), R₅ being chosen from the compounds corresponding to the formula R₄Ph, R₄ being chosen from H—, NO₂—, CH₃O—, CN—, Cl—, Br— and F—, and R₅ possibly also being chosen from the group consisting of: CH₃—, ClCH₂—, Cl₂CH—, Cl₃C— and CH₃CH₂—;
R represents the hydrogen atom or a group of formula (IIa):
   (HO)ₘ—OC—(X)—CO— (IIa)
   in which:

m represents 0 when n=2 and m represents 1 when n=1,

X represents a single bond or a group chosen from —(CH₂)—, saturated or unsaturated, linear, branched or cyclic C₂-C₂₀ alkyls, C₆-C₂₀ aryls and C₈-C₂₀ aralkyls.

A subject of the invention is the compounds of formula (A) defined above, with the exclusion of:
those for which R=H,
those for which n=1, Z₁, Z₂ represents (H,H) and X represents:
   —(CH₂)₂— or —(CH₂)₆—,
   and
those for which n=2, Z₁, Z₂ represents (H,H) and X represents:
   —(CH₂)—, —(CH₂)₂— —(CH₂)₃— or —(CH₂)₄—.

According to the present invention, the compounds corresponding to formula (VII): 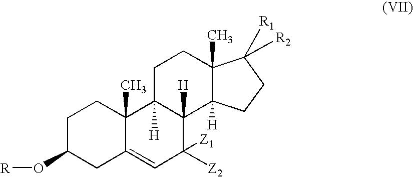
in which
R₁, R₂ represent ═O or a group —O—W—O— in which W represents a saturated or unsaturated, linear, branched or cyclic C₂-C₈ alkyl group; preferably, R₁, R₂ represents —O—CH₂—CH₂—O—,
R represents a group of formula (IIb)
   HO—OC—(X)—CO— (IIb)
   in which X represents a single bond or a group chosen from —(CH₂)—, saturated or unsaturated, linear, branched or cyclic C₂-C₂₀ alkyls, C₆-C₂₀ aryls and C₈-C₂₀ aralkyls,
Z₁, Z₂ represent ═O, (H, —OH) or (H,H), and which correspond to formula (A) when n=1, can be used for the preparation of other active compounds that can be used in particular in cosmetics.

The compounds of formula (VII) correspond to the hemiesters (in the case where n=1) of formulae (III), (IV), (V) and (VI).

The compounds of formula (VII) comprise a carboxylic acid function that can be used to carry out a coupling to another molecule that can be chosen in particular from cosmetic active principles comprising at least one function capable of forming a covalent bond with the carboxylic acid function. This is the case, for example, of retinol, of α-hydroxy acids and of α-keto acids.

In general, a cosmetically or dermatologically active molecule comprising at least one alcohol function or an amine function is grafted to the molecule (VII) by its carboxylic acid function, so as to form either an ester function or an amide function. If the active molecule comprises other functionalities capable of reacting during coupling with the molecule (VII), they are advantageously protected using an appropriate protective group according to methods well known to those skilled in the art (the case of α-hydroxy acids, for example).

When the molecule (VII) also comprises a functionality capable of interfering with this coupling reaction, said functionality is advantageously protected using an appropriate protective group (in the case where (Z₁, Z₂)=(H,OH)).

This reaction advantageously gives the molecules of formula (VIII): 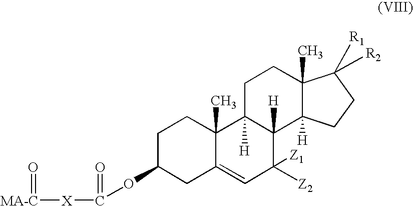
in which R₁, R₂, Z₁, Z₂ and X have the same meaning as above, and MA denotes a cosmetically active molecule, for instance retinol for its cosmetic properties (anti-wrinkle, anti-aging properties), α-hydroxy acids or α-keto acids for their exfoliant properties, α-bisabolol for its anti-inflammatory properties, or trans-farnesol for its bacteriostatic properties, α-tocopherol for its antioxidant properties, and amino acids such as in particular natural amino acids.

A subject of the invention is also the cosmetic and/or dermatological compositions comprising at least one compound of formula (VIII) in a cosmetically and/or dermatologically acceptable carrier.

Such compositions are intended in particular to prevent and/or delay and/or treat the appearance of signs of skin aging.

### EXPERIMENTAL SECTION

### Preparation of 3β-hydroxy-17,17-ethylenedioxy DHEA

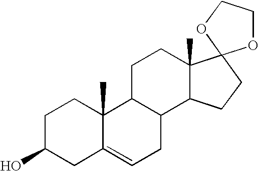

Para-toluenesulfonic acid (0.29 g, 1.5 mmol) and ethylene glycol (65 ml, 1.17 mol) are added to a solution of DHEA (50 g, 0.173 mol) in toluene (170 ml), placed in a three-necked flask surmounted with a Dean Stark apparatus and a condenser, and the entire mixture is brought to reflux. The reaction mixture is left for 4 hours with stirring and allowed to return to ambient temperature, and then sodium bicarbonate (100 mg) is added. The residual ethylene glycol is removed by simple separation by settling out, and the organic phase is concentrated. The resulting oil is taken up with ethyl acetate (300 ml). The organic phase is washed twice with a saturated sodium chloride solution and twice with water. After drying over MgSO₄, it is concentrated and the precipitate obtained is recrystallized in a minimum amount of ethanol.

Rf 0.19 (8:2 hexane/ethyl acetate) Melting point: 164-166° C. IR: 3500-3300 cm⁻¹, v OH free and bound; 2900 cm⁻¹, v CH₂; 1660 cm⁻¹, v C═C ¹H NMR (CDCl₃, 200 MHz): δ 5.35 ppm (d, 1H, H at C-6), 3.95 ppm (dd, 4H, H from dioxolane), 3.55 ppm (m, 1H, H at C-3), 1.15 to 2.4 ppm (m, 19H, H at C-1, C-2, C-4, C-7, C-8, C-9, C-11, C-12, C-14, C-15, C-16), 1.05 ppm (s, 3H, H at C-19), 0.9 ppm (s, 3H, H at C-18).

### A—Hemi-esterification of DHEA using anhydrides (succinic, glutaric, maleic, suberic and phthalic anhydride)

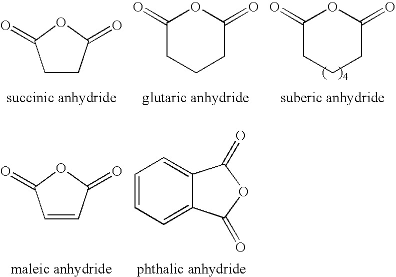

The coupling between the suberic anhydride and the DHEA is described in the article:
J. Org Chem, 1987, 52(16), 3573-3578

The coupling between the succinic anhydride and the DHEA is described in the article:
Steroids, 1998, 63(3), 158-165

### 1—First Method

Dimethylaminopyridine (DMAP) (12.7 g-3 eq-104.09 mmol) and succinic anhydride (10.41 g/9.63 ml-3 eq-104.09 mmol) are added to a solution of DHEA (10 g-1 eq-34.69 mmol) in 100 ml of dichloromethane. The reaction mixture is stirred at ambient temperature for 12 hours and then poured into water and extracted 3 times with dichloromethane.

The organic phase is washed with a 5% HCl solution and with a saturated solution of sodium salt, dried over sodium sulfate, and then concentrated under vacuum.

The resulting crude is purified by recrystallization from the mixture methanol/acetone, to give the corresponding hemiester with a yield of 68%.

Rf: 0.36 6:4:0.1 hexane/AcOEt/Ac acetic acid ¹H NMR (CDCl₃, 200 MHz): δ 5.4 ppm (d, 1H, H at C-6), 4.6 ppm (m, 1H, H at C-3), 2.65 ppm (dd, 4H, H from succinate), 1.15 to 2.6 ppm (m, 19H, H at C-1, C-2, C-4, C-8, C-9, C-11, C-12, C-14, C-15, C-16), 1.05 ppm (s, 3H, H at C-19), 0.9 ppm (s, 3H, H at C-18).

The glutaric acid hemiester is obtained in a similar manner, with a yield of 54%.

¹H NMR (CDCl₃, 200 MHz): δ 5.4 ppm (d, 1H, H at C-6), 4.6 ppm (m, 1H, H at C-3), 2.65 ppm (dd, 4H), 1.67 ppm (m, 4H), 1.15 to 2.6 ppm (m, 19H, H at C-1, C-2, C-4, C-8, C-9, C-11, C-12, C-14, C-15, C-16), 1.05 ppm (s, 3H, H at C-19), 0.9 ppm (s, 3H, H at C-18).

The maleic acid hemiester is obtained after purification by silica gel column chromatography (6:4:0.1-hexane/EtOAc/AcOH). 85% yield.

¹H NMR (CDCl₃, 200 MHz) : δ 6.45 ppm (d, 1H, H from maleic anhydride), 6.35 ppm (d, 1H, H from maleic anhydride), 5.45 ppm (d, 1H, H at C-6, J₆₇=4.8 Hz), 4.8 ppm (m, 1H, H at C-3), 1.15 to 2.6 ppm (m, 21H, H at C-1, C-2, C-4, C-7, C-8, C-9, C-11, C-12, C-14, C-15, C-16).

The 1,2-benzoic acid hemiester is obtained in a similar manner, with a yield of 47%.

¹H NMR (CDCl₃, 200 MHz): δ 8.25 ppm (dd, 1H,), 8.20 ppm (dd, 1H,), 7.71 ppm (m, 2H), 5.45 ppm (d, 1H, H at C-6, J₆₇=4.8 Hz), 4.8 ppm (m, 1H, H at C-3), 1.15 to 2.6 ppm (m, 21H, H at C-1, C-2, C-4, C-7, C-8, C-9, C-11, C-12, C-14, C-15, C-16).

### 2—Second Method

Succinic anhydride (17.35 g/16.06 ml-5 eq-173.48 mmol) is added to a solution of DHEA (10 g-1 eq-34.69 mmol) in 50 ml of pyridine (freshly distilled) . The reaction mixture is stirred at ambient temperature for 12 hours and then poured into water and extracted 3 times with dichloromethane.

The organic phase is washed with a 5% HCl solution and with a saturated solution of sodium salt, dried over sodium sulfate, then concentrated under vacuum. The resulting crude is purified by recrystallization from the mixture methanol/acetone, to give the corresponding hemiester with a yield of 72%.

The suberic acid hemiester is obtained in a similar manner, after silica gel column chromatography (ether), and is then recrystallized from the EtOAc/hexane system. Yield=39%.

### B—Hemi-esterification of DHEA using diacids

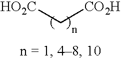

Adipic acid (5 g-1 eq-34.21 mmol) in solution in 50 ml of pyridine is treated with para-toluenesulfonyl chloride (11.91 g-0.9 eq-31.2 mmol). The entire mixture is left at 0° C. for 30 minutes, with stirring. DHEA (2.81 g-0.28 eq-9.77 mmol) in solution in 60 ml of pyridine is added dropwise to the resulting reaction mixture. After stirring at ambient temperature for 4 hours, 250 ml of water are added to the reaction mixture and the entire mixture is extracted three times with ethyl acetate. The organic phase is washed with a 5% HCl solution and with a saturated solution of sodium salt, dried over sodium sulfate, and then concentrated under vacuum. The resulting crude is purified by silica gel column chromatography (6:4:0.1 hexane/EtOAc/AcOH), to give the corresponding hemiester with a yield of 92%.

The other diacids are treated in the same manner, to give the corresponding hemiesters, virtually quantitatively.

### C—Preparation of the diesters of DHEA

### 1—First Method: using oxalyl chloride

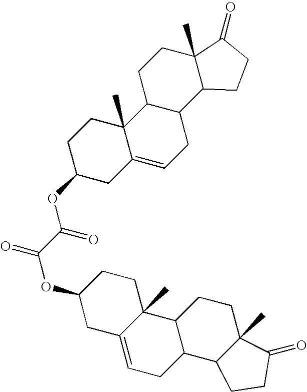

Oxalyl chloride (1.09 g-0.5 eq-8.67 mmol) is added dropwise, at 0° C., to a solution of DHEA (5 g-1 eq-17.34 mmol) in 20 ml of pyridine. The reaction mixture is stirred at ambient temperature for 2 hours and then poured into water. The precipitate is washed with water and then with heptane.

The resulting crude is purified by silica gel column chromatography (8:2 hexane/AcOEt). Yield: 54%.

¹H NMR (CDCl₃, 200 MHz): δ 5.4 5 ppm (d, 2H, H at C-6 and at C-6′), 4.75 ppm (m, 2H, H at C-3 and at C-3′), 1.15 to 2.6 ppm (m, 38H, H at C-1, C-1′, C-2, C-2′, C-4, C-4′, C-7, C-7′, C-8, C-8′, C-9, C-9′, C-11, C-11′, C-12, C-12′, C-14, C-14′, C-15, C-15′, C-16 and C-16′), 1.05 ppm (s, 6H, H at C-19 and C-19′), 0.9 ppm (s, 6H, H at C-18 and C-18′).

Mass spectrum: FAB⁺ [M+H]⁺=631 and [M+Na]⁺=653.

### 2—Second Method: Using the hemiesters prepared in § A and B

This methodology is based on esterification of the 3-position of DHEA with DHEA hemiesters in the presence of N,N′-carbonyldiimidazole.

### EXAMPLE

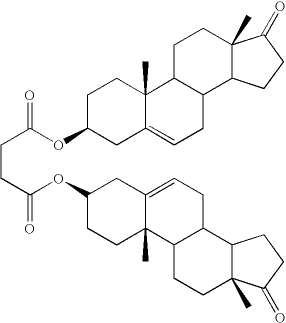

The DHEA hemisuccinate (1 g-1 eq-2.57 mmol) and the N,N′-carbonyldiimidazole (0.83 g-2 eq-5.14 mmol) are solubilized in 60 ml of anhydrous THF and the entire mixture is left at ambient temperature with stirring for 12 hours. The DHEA (3.7 g-5 eq-12.85 mmol) is then added and the entire mixture is then brought to reflux for eight hours.

The reaction mixture is subsequently cooled, diluted with 200 ml of water, and then extracted with chloroform. The organic phase is washed with water, dried with magnesium sulfate, and then concentrated under vacuum. The resulting crude is purified by silica gel column chromatography (97.5/2.5 hexane/AcOEt), and then recrystallized from methanol, to give the corresponding diester with a yield of 24%.

Rf: 0.32 9/1: hexane/ethyl acetate. ¹H NMR (CDCl₃, 200 MHz): δ 5.4 ppm (dd, 2H, H at C-6 and at C-6′), 3.75 ppm (m, 2H, H at C-3 and at C-3′), 2.55 ppm (dd, 4H, H from succinate), 1.1 to 2.45 ppm (m, 38H, H at C-1, C-1′, C-2, C-2′, C-4, C-4′, C-7, C-7′, C-8, C-8′, C-9, C-9′, C-11, C-11′, C-12, C-12′, C-14, C-14′, C-15, C-15′, C-16 and C-16′), 1.05 ppm (s, 6H, H at C-19 and C-19′), 0.9 ppm (s, 6H, H at C-18 and C-18′).

As an alternative to this technique, the dimer can be obtained by treatment of the DHEA hemiester with thionyl chloride and DHEA.

Thionyl chloride (0.9 g-3 eq-7.71 mmol) is added to a solution of DHEA hemisuccinate (1 g-1 eq-2.57 mmol) in 20 ml of carbon tetrachloride. The entire mixture is brought to reflux for 2 to 3 hours. After cooling, the medium is concentrated under vacuum. The resulting crude is treated with DHEA (1.11 g-1.5 eq-3.855 mmol) in solution in 50 ml of dichloromethane, and is then left at ambient temperature overnight, with stirring.

The reaction medium is poured into a 5% aqueous sodium bicarbonate solution. The two phases are separated, and the aqueous phase is washed with 100 ml of dichloromethane. The organic phases are combined, washed with water, dried over sodium sulfate, and then concentrated under vacuum. The resulting crude is purified by silica gel column chromatography (97.5/2.5 hexane/AcOEt) and then recrystallized from methanol, to give the corresponding diester with a yield of 32%.

### D—Allylic Oxidation

### 1) By Photooxidation

### Preparation of 3β,3β′-O-oxalylbis(7-oxo-17,17-ethylene-dioxy DHEA)

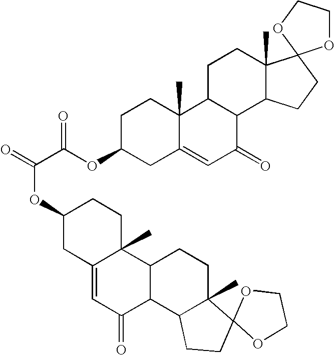

3β,3β′-O-oxalylbis(17,17-ethylenedioxy)-DHEA (5 g-6.95 mmol) is solubilized in 50 ml of pyridine in the presence of rose Bengal (30 mg) . The reaction mixture is irradiated with a sodium lamp (400 w) under a continuous stream of compressed air, with stirring and with a refrigeration system regulated at 10° C. After 96 hours, the reaction mixture is cooled and acetic anhydride (7.5 ml-0.166 mol) is added. The reaction is left at ambient temperature overnight, with stirring. At the end of the reaction, the pyridine is concentrated under vacuum. The resulting crude is taken up with ethyl acetate (20 ml) and then washed with water and then with a saturated sodium chloride solution, and the organic phase is dried over magnesium sulfate and then concentrated under vacuum. Recrystallization from a minimum amount of ethanol makes it possible to obtain the 3β,3β′-O-oxalylbis(7-oxo-17,17-ethylenedioxy)-DHEA with a yield of 43%.

¹H NMR (CDCl₃, 250 MHz): δ 5.7 ppm (d, 2H, H at C-6), 3.75 ppm (m, 2H, H at C-3), 3.9 ppm (s, 8H, H from dioxolane), 1.3 to 2.5 ppm (m, 34H, H at C-1, C-2, C-4, C-7, C-8, C-9, C-11, C-12, C-14, C-15, C-16), 1.25 ppm (s, 6H, H at C-19), 0.9 ppm (s, 6H, H at C-18).

Mass spectrum: FAB⁺ [M+H]⁺=747 and [M−Na]⁺=769. ¹H NMR (CDCl₃, 200 MHz): δ 5.7 ppm (d, 1H, H at C-6), 3.7 ppm (m, 1H, H at C-3), 1.3 to 2.9 ppm (m, 17H, H at C-1, C-2, C-4, C-8, C-9, C-11, C-12, C-14, C-15, C-16), 1.2 ppm (s, 3H, H at C-19), 0.9 ppm (s, 3H, H at C-18).

### 2) Oxidation using N-hydroxyphthalimide

3β-O-acetyl-17,17-ethylenedioxy-DHEA (50 g-134 mmol) in solution in an ethyl/ acetate/acetone mixture (250/250 ml) is placed in a reactor equipped with a condenser. After addition and solubilization of the N-hydroxyphthalimide (21.8 g, 9.38 mmol), the reaction medium is added and brought to reflux for 5 days under a constant stream of compressed air. At the end of the reaction, the solvents are concentrated and the medium is taken up in cold toluene. The N-hydroxyphthalimide precipitate is filtered off and the filtrate is washed with a saturated sodium bicarbonate solution, with a sodium chloride solution, and then finally with water. The organic phase is dried over magnesium sulfate and then concentrated under vacuum. The resulting crude is taken up with pyridine (150 ml) and is then added dropwise to acetic anhydride (75 ml). The reaction is left at ambient temperature for 15 hours, with stirring. At the end of the reaction, the pyridine is concentrated under vacuum, the resulting crude is taken up with ethyl acetate (100 ml), and the organic phase is washed twice with water and twice with a saturated sodium chloride solution, dried over magnesium sulfate, and then concentrated under vacuum. Recrystallization from a minimum amount of methanol makes it possible to obtain the desired product with a yield of 44%.

¹H NMR (CDCl₃, 250 MHz): δ 5.7 ppm (d, 1H, H at C-6), 3.75 ppm (m, 1H, H at C-3), 3.9 ppm (s, 4H, H from dioxolane), 2.1 ppm (s, 3H, —O—CO—CH₃), 1.3 to 2.5 ppm (m, 17H, H at C-1, C-2, C-4, C-8, C-9, C-11, C-12, C-14, C-15, C-16), 1.25 ppm (s, 3H, H at C-19), 0.9 ppm (s, 3H, H at C-18).

### E—Deprotection

### 1—Deprotection of the 7-oxo derivatives

The compounds of formula (IV) are, firstly, deprotected by treatment with sodium methanolate in methanol, to give the 3β-hydroxy-7-oxo-17-dioxolane DHEA derivative

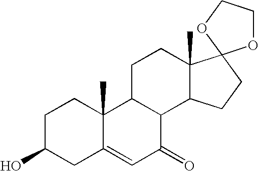

### 2—Deprotection of the 3,3′-oxalylbis(7-oxo-17,17-ethylenedioxy-DHEA) diester

3β,3β′-oxalylbis(7-oxo-17,17-ethylenedioxy-DHEA) (5 g-6.7 mmol) is taken up with methanol (50 ml), the entire mixture is cooled to 4° C., and sodium methanolate (MeONa) (0.76 g-20 mmol) is added. After 4 hours, water is added and extraction is carried out with ethyl acetate (100 ml). The organic phase is washed several times with a saturated sodium chloride solution, dried over magnesium sulfate, and then concentrated under vacuum, to give the expected product after recrystallization from a minimum amount of ethanol.

Rf=0.68 (ethyl acetate) Yield 34%. ¹H NMR (CDCl₃, 250 MHz): δ 5.7 ppm (d, 1H, H at C-6), 3.9 ppm (m, 4H, H from the dioxolane function), 3.7 ppm (m, 1H, H at C-3), 1.3 to 2.8 ppm (m, 17H, H at C-1, C-2, C-4, C-8, C-9, C-11, C-14, C-15, C-16), 1.2 ppm (s, 3H, H at C-19), 0.9 ppm (s, 3H, H at C-18).

In a second step, the 17-position is deprotected to give 3β-hydroxy-7-oxo-DHEA.

The 3β-hydroxy-17,17-ethylenedioxy-DHEA derivative (1.5 g-0.43 mmol) is dissolved in 38 ml of acetone and then 67.5 ml of water and 21.5 ml of an aqueous solution containing 0.1% of perchloric acid are added. The reaction mixture is left at ambient temperature for 20 hours, with stirring. At the end of the reaction, a 5% sodium bicarbonate solution is added (100 ml). The acetone is driven off under vacuum and the aqueous phase is extracted with dichloromethane (3×10 ml). The organic phase is concentrated under vacuum and the resulting crude is recrystallized from methanol, to give the desired product with a yield of 65%.

### F—Diastereoselective Reduction

### 1—Obtaining 7α-hydroxylated derivatives

### Preparation of 3β,3β′-oxalylbis(7α-hydroxy-17,17-ethylene-dioxy-DHEA)

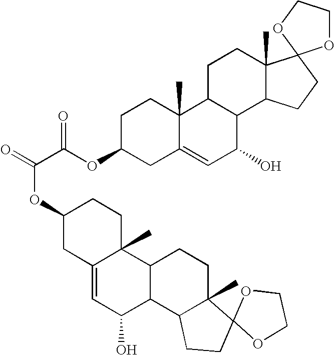

14.74 ml of L-Selectride® (1M in THF) are added dropwise, under an inert atmosphere, to a solution of 3β,3β′-oxalylbis(7-oxo-17,17-ethylenedioxy-DHEA) (5 g-6.7 mmol) in 60 ml of THF cooled to −78° C. After reaction for 2 hours, the reaction mixture is brought to 0° C. and water is added slowly with stirring. The medium is diluted with ether. The aqueous and organic phases are separated, and the organic phase is then washed with a saturated sodium chloride solution and dried over magnesium sulfate. The resulting crude product can be used as it is for the step of saponification with sodium methanolate, or can be purified by silica gel column chromatography (7/3: hexane/ethyl acetate).

¹H NMR (CDCl₃, 250 MHz): δ 5.65 ppm (d, 2H, H at C-6), 3.75 ppm (m, 2H, H at C-3), 3.9 ppm (s, 10H, H dioxolane+H-7), 1.3 to 2.5 ppm (m, 34H, H at C-1, C-2, C-4, C-8, C-9, C-11, C-12, C-14, C-15, C-16), 1.25 ppm (s, 6H, H at C-19), 0.9 ppm (s, 6H, H at C-18).

### Deprotection of the 3- and 17-Positions

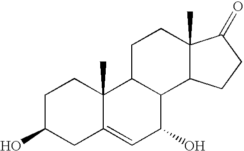

The 3β,3β′-oxalylbis(7α-hydroxy-17,17-ethylenedioxy-DHEA) is deacetylated according to sodium methanolate treatment already described, to give the 3β-hydroxy-7α-hydroxy-17,17-ethylenedioxy-DHEA derivative which, after treatment, is used in the final deprotection with perchloric acid to give the 3β-hydroxy-7α-hydroxy-DHEA with a yield of 54% (from the 7-oxo derivative).

Rf: 0.17 (7:3 hexane/ethyl acetate) Melting point: 187-189° C. HPLC: 9 min (gradient: 15/20/65 acetonitrile/-methanol/water to 100 acetonitrile) ¹H NMR (CDCl₃, 250 MHz): δ 5.6 ppm (d, 1H, H at C-6), 3.9 ppm (t, 1H, H at C-7α), 3.5 ppm (m, 1H, H at C-3), 1.1 to 2.6 ppm (m, 17H, H at C-1, C-2, C-4, C-8, C-9, C-11, C-12, C-14, C-15, C-16), 1.05 ppm (s, 3H, H at C-19), 0.9 ppm (s, 3H, H at C-18).

### 2—Obtaining 7β-hydroxylated derivatives

### Preparation of 3β,3β′-oxalylbis(7β-hydroxy-17,17-ethylene-dioxy-DHEA)

A solution of CeCl₃. 7 H₂O (2.49 g-6.7 mmol) in methanol (32.5 ml) is added dropwise to a solution of 3β,3β′-oxalylbis(7-oxo-17,17-ethylenedioxy-DHEA) (5 g-6.7 mmol) in THF (14 ml), cooled to −5° C. After stirring for approximately 5 minutes, NaBH₄ (0.5 g-13.4 mmol) is added in several steps. After reaction at −5° C. for 15 minutes, acetone (15 ml) is slowly added. The reaction medium is allowed to return to ambient temperature and is then concentrated under vacuum. The resulting crude can be used as it is for the step of saponification with sodium methanolate, or can be purified by silica gel column chromatography (7/3: hexane/ethyl acetate).

### Deprotection of the 3- and 17-Positions

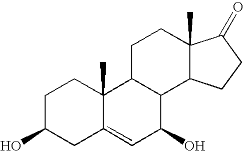

The 3β,3β′-oxalylbis(7β-hydroxy-17,17-ethylenedioxy-DHEA) is deacetylated according to the sodium methanolate treatment already described, to the 3β-hydroxy-7β-hydroxy-17,17-ethylenedioxy-DHEA derivative which, after the final deprotection with perchloric acid, gives the 3β-hydroxy-7β-hydroxy-DHEA with a yield of 58% (from the 7-oxo derivative).

Rf: 0.27 (7:3 hexane/ethyl acetate). HPLC: 7.7 min (gradient: 15/20/65 acetonitrile/-methanol/water to 100 acetonitrile). ¹H NMR (CDCl₃, 200 MHz): δ 5.3 ppm (d, 1H, H at C-6), 3.95 ppm (dd, 1H, H at C-7β), 3.55 ppm (m, 1H, H at C-3), 1.15 to 2.55 ppm (m, 17H, H at C-1, C-2, C-4, C-8, C-9, C-11, C-12, C-14, C-15, C-16), 1.1 ppm (s, 3H, H at C-19), 0.9 ppm (s, 3H, H at C-18).

### G—Inversion of Configuration of 7β to 7α

### 1—Mitsunobu Reaction

### Preparation of 17,17-ethylenedioxy-DHEA 3β-O-hemisuccinate 7β-O-(p-nitro)benzoate

A solution of diethyl azodicarboxylate (3.71 ml-23.75 mmol) is added dropwise, at ambient temperature, to a solution of 7β-hydroxy-17,17-ethylenedioxy-DHEA 3β-O-hemisuccinate (2 g-4.75 mmol), of triphenylphosphine (6.3 g-23.75 mmol) and of para-nitrobenzoic acid (3.95 g-23.75 mmol) in benzene (50 ml). The medium is left for 18 hours with stirring. The solvent is then concentrated under vacuum and the resulting crude is purified by silica gel column chromatography (8:2:0.1 hexane/ethyl acetate/acetic acid). Yield 34%.

¹H NMR (CDCl₃, 200 MHz): δ 8.35 ppm (dd, 2H, H from the aromatic ring), 8.24 ppm (dd, 3H, H from the aromatic ring), 5.75 ppm (d, 1H, H at C-6), 5.35 ppm (dd, 1H, H at C-7), 4.65 ppm (m, 1H, H at C-3), 1.15 to 2.5 ppm (m, 17H, H at C-1, C-2, C-4, C-8, C-9, C-11, C-12, C-14, C-15, C-16), 1.1 ppm (s, 3H, H at C-19), 0.9 ppm (s, 3H, H at C-18).

Deprotection of the 3- and 7-positions in a methanolic medium, followed by deprotection of the 17-position, gives 3β-hydroxy-7α-hydroxy-DHEA with a yield of 72%.

### 2—Walden Inversion

### Preparation of 17,17-ethylenedioxy-DHEA 3β-O-hemisuccinate 7β-O-mesyl

Methanesulfonyl chloride (11.06 g-96.58 mmol) is added to a solution of 17β-hydroxy-17,17-ethylenedioxy-DHEA 3β-O-hemisuccinate (2 g-4.75 mmol) in dichloromethane (100 ml) (triethylamine (13.5 ml-96.58 mmol). The medium is left at 4° C. for 5 days, with stirring. The solvent is concentrated under vacuum and then taken up with a methanol/water/THF mixture (3/10/10-50 ml) to which KOH (5 g-100 mmol) is added. The entire mixture is brought to reflux for 24 hours. After having been allowed to return to ambient temperature, the medium is concentrated under vacuum so as to drive off the volatile solvents, rediluted with water, and then extracted with dichloromethane. The organic phase is washed with water, with a 5% aqueous acetic acid solution and then with a saturated aqueous sodium chloride solution. The organic phase is dried over magnesium sulfate, filtered, and then concentrated under vacuum. The resulting crude is used directly in the step consisting of deprotection of the 17-position, to give 3β-hydroxy-7α-hydroxy-DHEA after silica gel column chromatography (1:9 then 0:10 hexane/ethyl acetate). Yield 23%.

1. A compound characterized in that it corresponds to formula (A): 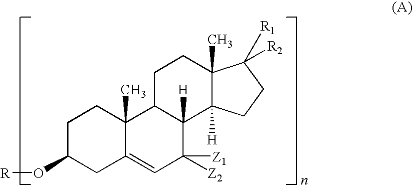
in which
n=1, 2,
R₁, R₂ represent ═O or a group —O—W—O— in which W represents a saturated or unsaturated, linear, branched or cyclic C₂-C₈ alkyl group; preferably, W represents —CH₂—CH₂—,
Z₁, Z₂ represent ═O, (H,OH) or (H,H), (H,R₅CO₂—), R₅ being chosen from the compounds corresponding to the formula R₄Ph, R₄ being chosen from H—, NO₂—, CH₃O—, CN—, Cl—, Br— and F—, and R₅ possibly also being chosen from the group consisting of: CH₃—, ClCH₂—, Cl₂CH—, Cl₃C— and CH₃CH₂—;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls, it being understood that, when n=1 and Z₁, Z₂ represents (H,H), X is different from —(CH₂)₂ and from —(CH₂)₆— and, when n=2 and Z₁, Z₂ represents (H,H), X is different from —(CH₂)—, from —(CH₂)_{2—}, from —(CH₂)₃— and from —(CH₂)₄—.

2. A method for synthesizing DHEA derivatives chosen from 7α-OH-DHEA, 7β-OH-DHEA and 7-oxo-DHEA, this method using DHEA as starting product, this method being characterized in that:
(1) in a first step, the ketone function in the 17-position of the DHEA is optionally protected with a protective group;
(2) in a second step, the hemiester (n=1) or the diester (n=2) is prepared between the alcohol in the 3-position of the compound obtained in step (1) and a dicarboxylic acid chosen from those corresponding to formula (II):
HOOC—X—COOH (II)
so as to obtain a compound as claimed in claim 1, corresponding to formula (III): 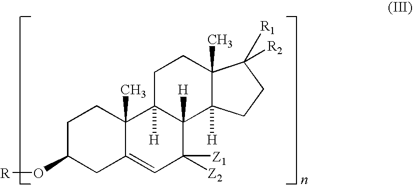
in which:
n represents an integer chosen from 1 and 2;
R₁, R₂ represent a group ═O or a group —O—W—O— in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

3. The method as claimed in claim 2, characterized in that the DHEA is protected in the 17-position in the form of a cyclic acetal by treatment with ethylene glycol, at the reflux of toluene, in the presence of para-toluenesulfonic acid using a Dean Stark apparatus.

4. The method as claimed in either one of claims 2 and 3, characterized in that the dicarboxylic acid is chosen from:
oxalic acid, succinic acid, glutaric acid, suberic acid, maleic acid, phthalic acid; isophthalic acid, terephthalic acid, 1,2-phenylenediacetic acid, 1,3-phenylenediacetic acid and 1,4-pheny-lenediacetic acid.

5. The product as claimed in claim 1, which can be obtained by means of the method as claimed in any one of claims 2 to 4, characterized in that it corresponds to formula (III): 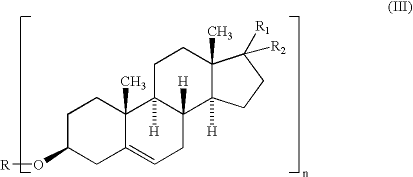
in which:
n represents an integer chosen from 1 and 2;
R₁, R₂ represent a group ═O or a group —O—W—O— in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls, it being understood that, when n=1, X is different from —(CH₂)₂— and from —(CH₂)₆— and, when n=2, X is different from —(CH₂)—, from —(CH₂)₂—, from —(CH₂)₃— and from —(CH₂)₄—.

6. The method as claimed in any one of claims 2 to 4, characterized in that it also comprises a step of allylic oxidation on the carbon in the 7-position of the compound corresponding to formula (III), so as to obtain the ketone of formula (IV): 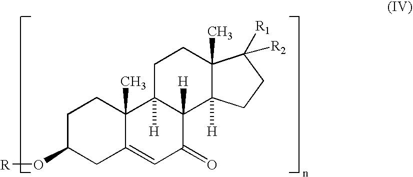
in which:
n represents an integer chosen from 1 and 2;
R₁, R₂ represent a group ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

7. The method as claimed in claim 6, characterized in that the compound of formula (III) is treated by photooxidation by means of a lamp and with sparging with oxygen or with compressed air in the presence of rose Bengal, followed by treatment with acetic anhydride in pyridine.

8. The method as claimed in claim 6, characterized in that the compound of formula (III) is treated with N-hydroxyphthalimide with sparging with oxygen or with compressed air.

9. The method as claimed in any of one of claims 6 to 8, for preparing 7-oxo-DHEA, characterized in that it also comprises a step during which the compound of formula (IV) is treated so as to remove the protective groups from the alcohol in the 3-position and, optionally, from the ketone in the 17-position.

10. The compound as claimed in claim 1, which can be obtained by means of the method as claimed in any one of claims 6 to 8, characterized in that it corresponds to formula (IV): 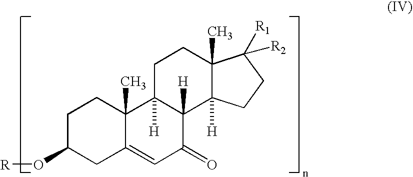
in which:
n=1, 2,
R₁, R₂ represent ═O or a group —O—W—O—, in which W represents a saturated or unsaturated, linear, branched or cyclic C₂-C₈ alkyl group,
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

11. The method as claimed in claim 6, for preparing a DHEA derivative, characterized in that it also comprises a step during which the compound of formula (IV) is diastereoselectively reduced to give the 7β-OH derivative corresponding to formula (V): 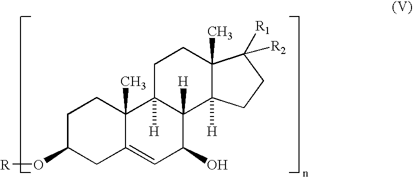
in which:
n represents an integer chosen from 1 and 2;
R₁, R₂ represent a group ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

12. The method as claimed in claim 11, characterized in that the reduction is carried out by treatment with NaBH₄ in the presence of cerium chloride.

13. The method as claimed in claim 11 or claim 12, for preparing 7β-OH-DHEA, characterized in that it also comprises a step during which the acetal in the 17-position and the ester in the 3-position of the compound of formula (V) are deprotected.

14. The compound as claimed in claim 1, which can be obtained by means of the method as claimed in any one of claims 11 to 13, characterized in that it corresponds to formula (V): 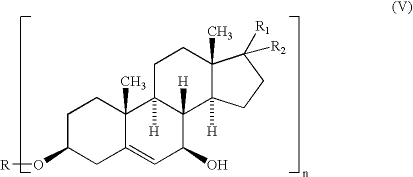
in which:
n=1, 2,
R₁, R₂ represent ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

15. The method as claimed in claim 11, for preparing a DHEA derivative, characterized in that it also comprises a step during which the compound of formula (V) is treated according to the Walden method or the Mitsunobu method, to give a 7α-OH compound corresponding to formula (VI): 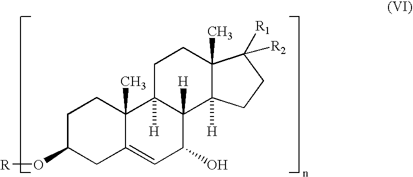
in which:
n represents an integer chosen from 1 and 2;
R₁, R₂ represent a group ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

16. The method as claimed in any one of claims 6 to 8, for preparing a DHEA derivative, characterized in that it comprises a step during which the compound of formula (IV) is diastereoselectively reduced to give the 7α-OH derivative corresponding to formula (VI): 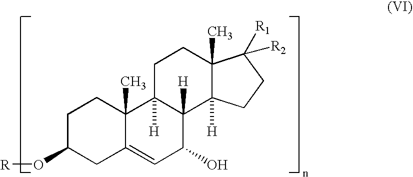
in which:
n represents an integer chosen from 1 and 2;
R₁, R₂ represent a group ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

17. The method as claimed in claim 16, characterized in that the reduction is carried out using lithium tri-sec-butylborohydride.

18. The method as claimed in claim 15 or claim 16, for preparing 7α-OH-DHEA, characterized in that it also comprises a step during which the acetal in the 17-position and the ester in the 3-position of the compound of formula (VI) are deprotected.

19. The compound as claimed in claim 1, which can be obtained by means of the method as claimed in claim 16 or claim 17, characterized in that it corresponds to formula (VI): 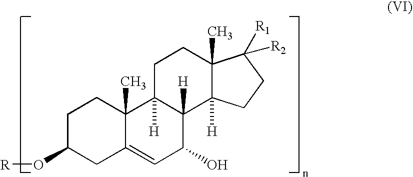
in which:
n=1, 2,
R₁, R₂ represent ═O or a group —O—W—O—, in which W represents a saturated or unsaturated, linear, branched or cyclic C₂-C₈ alkyl group;
R represents a group of formula (IIa)
(HO)ₘ—OC—(X)—CO— (IIa)
in which:
m represents 0 when n=2 and m represents 1 when n=1,
X represents a single bond or a group chosen from —(CH₂)—, saturated or unsaturated, linear, branched or cyclic C₂-C₂₀ alkyls, C₆-C₂₀ aryls and C₈-C₂₀ aralkyls.

20. A method for preparing a DHEA derivative, this method being characterized in that it comprises a step during which a compound corresponding to formula (VII): 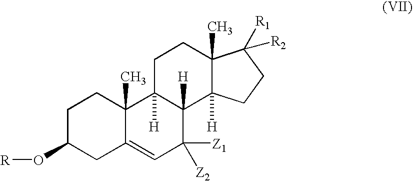
in which
R₁, R₂ represent ═O or a group —O—W—O— in which W represents a saturated or unsaturated, linear, branched or cyclic C₂-C₈ alkyl group; preferably, R₁, R₂ represents —O—CH₂—CH₂—O—,
R represents a group of formula (IIb)
(HO)—OC—(X)—CO— (IIb)
in which X represents a single bond or a group chosen from —(CH₂)—, saturated or unsaturated, linear, branched or cyclic C₂-C₂₀ alkyls, C₆-C₂₀ aryls and C₈-C₂₀ aralkyls,
Z₁, Z₂ represent ═O, (H, —OH) or (H,H), is reacted, via its carboxylic acid function, with a cosmetically or dermatologically active molecule comprising at least one alcohol function or one amine function, so as to form either an ester function or an amide function.

21. A compound which can be obtained by means of the method as claimed in claim 20, characterized in that it corresponds to formula (VIII): 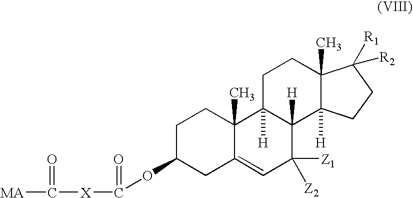
in which:
R₁, R₂ represent ═O or a group —O—W—O— in which W represents a saturated or unsaturated, linear, branched or cyclic C₂-C₈ alkyl group; preferably, R₁, R₂ represents —O—CH₂—CH₂—O—,
X represents a single bond or a group chosen from:
—(CH₂)—, saturated or unsaturated, linear, branched or cyclic C₂-C₂₀ alkyls, C₆-C₂₀ aryls and C₈-C₂₀ aralkyls,
Z₁, Z₂ represent ═O, (H, —OH) or (H,H),
MA denotes a cosmetically active molecule chosen from retinol, an α-hydroxy acid, an α-keto acid, α-bisabolol, or trans-farnesol, α-tocopherol and a natural amino acid.

22. A cosmetic and/or dermatological composition comprising at least one compound of formula (VIII) as claimed in claim 21, in a cosmetically and/or dermatologically acceptable carrier.

23. The use of a compound corresponding to formula (VIII) as claimed in claim 21, for preparing a cosmetic or dermatological composition intended to prevent and/or delay and/or treat the appearance of signs of skin aging.
